(19) United States
(12) Patent Application Publication
(10) Pub. No.: US 2008/0153144 A1
(43) Pub. Date: Jun. 26, 2008

(54) METHOD FOR CONVERTING HERBACEOUS PLANT FIBERS INTO FUEL ALCOHOL

(76) Inventors: Kaikwai CHAN, Hong Kong (CN); Wai HUI, Hong Kong (CN)

Correspondence Address: MATTHIAS SCHOLL, 14781 MEMORIAL DRIVE, SUITE 1319, HOUSTON, TX, 77079, US

(21) Appl. No.: 11/613,463

(22) Filed: Dec. 20, 2006

(51) Int. Cl. C12P 7/00

(52) U.S. Cl. 435/165

(57) ABSTRACT

This disclosure teaches a method of converting herbaceous plant fibers into fuel alcohol comprising the following steps: The pre-treatment stage consists of grinding; using ultrasonic waves; adding the liquids mixed with alcohol, liquid ammonia, and water; adding NaOH; and then stirring and cooking. The second stage involves the recovery of organic liquids as well as high-pressure and high-temperature washing. Next, biological enzyme hydrolysis is conducted by adding endo-β-glucanase, exo-β-glucanase, and β-glucanase. Candida mycoderma, Rhizopus oryzae, ammonium sulfate, and phosphoric acid are added during the fermentation process. Finally, the alcohol is refined from distillate spirits, with further refinement in an alcohol tower.

### BACKGROUND OF THE INVENTION

1. Field of the Invention

This invention relates to methods of preparing alcohol, mainly referred to as a method of converting herbaceous plant fibers into fuel alcohol.

2. Description of the Related Art

At present, foods such as sorghum and corn serve as the main raw materials for producing fuel alcohol. The technology of producing fuel alcohol from corn consist of grinding the corn, mixing it with water in the proportion of from 1:30 to 1:40 corn to water by weight, heating the mixture to between 70 and 80° C., precooking for 20 to 40 minutes, and then boiling for no less than 90 minutes using a boiling vessel under 141 to 145° C. and pressure of from 3.2 to 3.5 kg/cm². However, many problems arise in using foods for the production of alcohol including high cost, low output rate, and high energy consumption.

Besides using foods, trials also have been made to produce alcohol using straws. The technology of such a method may consist of the following steps: grinding—syrup discharge—saccharification—sugar liquor—alcoholic fermentation—yeast separation—distillation—fuel alcohol. However, this method also has limitations such as high energy consumption and low output rate. Due to technical reasons, producing alcohol with straws is still at the experimental stage, which makes it unable to realize industrial production. Therefore, large numbers of straws and herbaceous plants are used as fuels, forages, and fertilizers, leading to a waste of resources.

### SUMMARY OF THE INVENTION

The aim of this invention is to provide a method of converting herbaceous plant fibers into fuel alcohol. By adding cellulase and other materials, cellulose is hydrolyzed to reduce sugar, which then is fermented to produce fuel alcohol. This method improves the output and the speed of cellulose hydrolysis and overcomes the shortcomings of the present technology for fuel alcohol production.

The technical scheme of realizing this invention: this method comprises the following steps:

1. Pretreatment

a. Grind the raw materials of herbaceous plants into 100 to 180 mesh, and load them in a reaction tank.

b. Loosen and separate the structures between the walls of raw fibers by adding CO₂ with supersonic waves.

c. Add a liquid mixture of alcohol, liquid ammonia, and water to the raw material in the proportion of between 1:1.5 and 1:3 (the weight ratio of the raw material to the liquid). The alcohol accounts for 30-40%, and liquid ammonia accounts for 6-10%; the rest is water.

d. Add dilute alkali NaOH with 3-12% of the weight ratio of the raw material and stir.

e. Boil for 20 minutes to 2 hours at between 100 and 250° C. and the pressure of 2-6 Mpa.

f. Reduce the pressure to normal and reduce the temperature to between 30 and 40° C.

2. Recovery

a. Recover the organic liquids.

b. Wash them under high pressure and high temperature.

3. Biological enzyme hydrolysis

a. At 40 to 60° C. and pH 3.0 to 6.0, add the cellulase comprising endo-β-glucanase, exo-β-glucanase and β-glucanase. This accounts for 0.5 to 3% of the liquid weight

b. The enzymolysis process takes about 8 to 12 hours.

4. Fermentation

a. Under the temperature of 40-60° C. and pH 3.0-6.0, add liquid Candida mycoderma, Rhizopus oryzae, and dry yeast (compounded), which account for 3 to 8% of the liquid weight.

b. Ferment for 50-80 hours at the temperature of between 30 and 40° C.; and

c. Optionally, further add ammonium sulfate and phosphoric acid during this process to enhance fermentation.

5. Refinement

a. When the detected alcohol concentration reaches between 18 and 25 degrees, it will come into the preliminary process of refining alcohol from distillate spirits.

b. When the alcohol concentration reaches between 35 and 50 degrees, it will be sent to the alcohol tower for further refinement until the concentration reaches 95 degrees.

The technical scheme further includes:

The said cellulase further comprises glucosiduronate enzyme, acetyl enzyme, xylanase, β-xylanase, galactomannoglycan enzyme, and glucomannan enzyme.

The pressure/temperature relief process comprises transient decompression and transient cooling as well as real-time water-adding temperature relief.

The recovery of organic liquids comprises pretreatment using different alcohol, liquid ammonia and dilute alkali NaOH.

The beneficial results of this invention are characterized by such features as extensive sources of raw material, simple process, low cost, low energy consumption, high output rate, environment protection and partial replacement of oil, this invention can also convert herbaceous plant fibers into other chemical products and biochemical products.

### BRIEF DESCRIPTION OF THE DRAWINGS

The preferred embodiments of the present invention will hereinafter be described in more detail with reference to the accompanying drawings, in which:

FIG. 1 shows the raw material flow chart of this invention; and

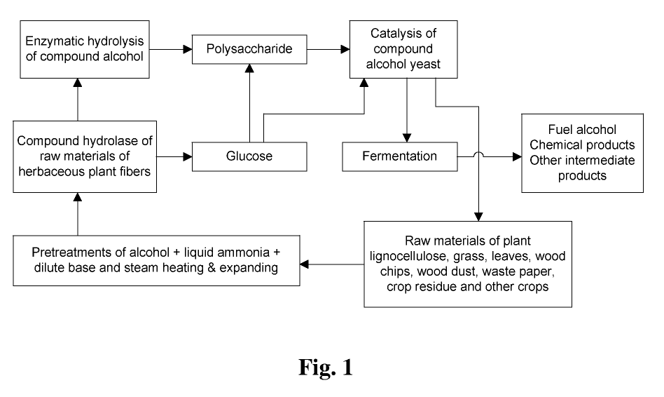

FIG. 2 shows the technical production flow chart of this invention.

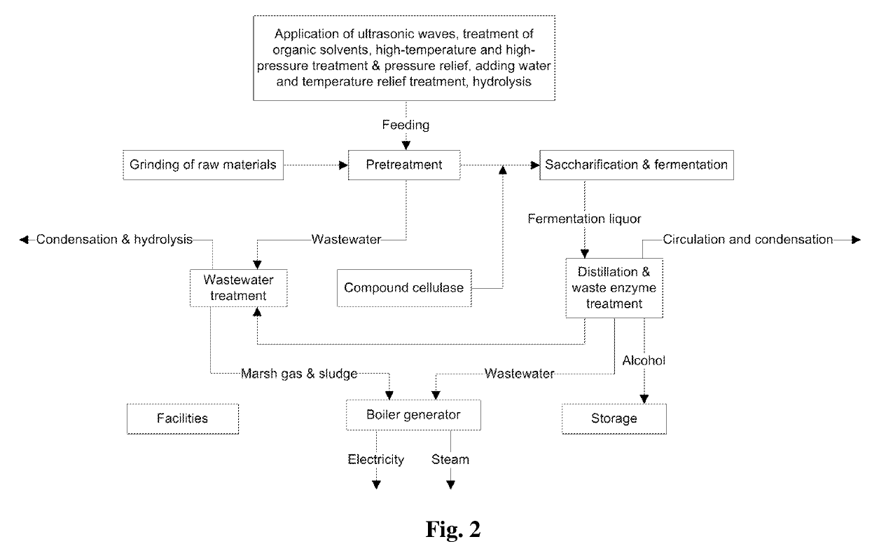

### DETAILED DESCRIPTION OF THE INVENTION

Herbaceous plant fibers exist extensively in nature as renewable energy resources. The fuel alcohol produced by applying enzyme hydrolysis to treat herbaceous plant fibers can partially replace oil. In addition, herbaceous plant fibers can be further converted into other chemical products and biochemical products. The following will describe the main technologies involved in the pretreatment process and the process of converting compound cellulase into fuel alcohol in this invention.

1. Brief description of producing alcohol with the raw materials of herbaceous plant lignocellulose

As a substitute energy source for limited oil resources, the raw materials of the herbaceous plant lignocellulose can be used to produce alcohol. Such herbaceous lignocelluloses can be found in crop residues, grasses, leaves, wood chips, wood dusts, waste papers, etc. As biological refineries of herbaceous plant fibers can replace oil refineries, there will be social and environmental benefits to converting the raw material of carbohydrates into hydrocarbons. In the alcohol production based on herbaceous plant fibers, the conversion process mainly comprises two parts: (i) the cellulose in the herbaceous plant fibers is hydrolyzed to generate reducing sugar; (ii.) the reducing sugar is fermented to produce alcohol. First, the raw materials of herbaceous plant fibers are pretreated with thermal physical and chemical treatment and are hydrolyzed with enzymes. Second, the pretreated raw materials are converted into fermentable sugars of alcohol. On one hand, the method of pretreatment concentrates on shortening the time for bio-transformation, reducing the dosage of cellulase, and improving the output rate of alcohol. On the other hand, the method of cellulose hydrolysis concentrates on utilizing the function of synergistic effect generated during the enzyme mixing so as to meet the demands for hydrolyzing cellulose to generate reducing sugar and fermenting reducing sugar to produce alcohol.

During the pretreatment of the raw materials of the herbaceous plant fibers, xylogen and hemicellulose can be removed, and the hydrolysis level of cellulose can be greatly improved; the application of appropriate cellulase can also improve the hydrolysis level. By realizing the technology of concurrent saccharification and fermentation, the glucose content restraining the activity of cellulase can be effectively reduced to improve the output and the speed of cellulose hydrolysis.

As the enzymic hydrolysis of herbaceous plant fibers features low energy consumption, product specificity, and is in accordance with environmental protection requirements, the herbaceous plant fiber will become one of the main forms of future energy sources.

2. Technology of producing alcohol from the raw materials of herbaceous plant lignocellulose

Pretreatment of the raw materials of herbaceous plant lignocellulose

The aim of pretreatment is to remove xylogen and hemicellulose, reduce crystallinity of cellulose, and increase porosity of the raw materials. The key factor of successful pretreatment is to destroy the structures of xylogen and hemicellulose. Effective pretreatment methods are analyzed as follows:

Most pretreatments cannot completely hydrolyze all celluloses in herbaceous plant fibers of the raw materials of xylem fibers. By removing the hemicelluloses around the micro-fibrous structures of cellulose, along with the changes of xylogen, pretreatment can make the enzymic hydrolysis of cellulose more effective. The elements in the pretreated raw materials of xylem fibers depend on the sources of herbaceous plant fibers and the types of the raw material to be pretreated. Under normal conditions, the hydrolysis of natural herbaceous plant fibers will be more effective if cellulase and relevant enzymes are used. The following gives a brief description for the technology of pretreating herbaceous plant fibers of grass type.

(1) Physical pretreatment of grinding powders: grind the raw materials into 100-180 mesh with grinding machine to reduce the size and mechanical lattice of herbaceous plant fiber granules.

(2) Physical pretreatment with ultrasonic waves: Loosen and separate the structural bonds between fiber walls of the raw materials by utilizing the principle of supersonic waves. Further loosen the fibrous structure chains between the fiber walls by adding CO₂.

(3) Chemical pretreatment with organic solvents: add 95% alcohol, liquid ammonia, and water to the reaction tank in the proportion of 40% to 6% to 54% by weight in sequence. Secondly, add 3 to 12% dilute alkali NaOH and mix with the water/material at a weight ratio of 1:2. Liquid ammonia and dilute alkali added together causes less destruction to cellulose and hemicellulose. The acetyl contained in the cellulose raw material is eliminated during the treatment with liquid ammonia; therefore, undesirable by-products will not occur in subsequent fermentation. This mechanism of pretreatment forms xylan as a result of saponification and cross-linking of intermolecular ester bonds. As the cross linkages are broken, the increase in porosity of the raw materials of xylem fiber results in inflation of the raw materials, which increases the internal surface area, decreases the degree of polymerization and crystallinity, separates and breaks the structural bonds between xylogen and carbohydrate, and completely destroys the xylogen structures.

(4) Pretreatment under high temperature and high pressure: increase the temperature to between 100 and 250° C. and the pressure to between 2 and 6 MPa with high-temperature steam; boil the mixture for between 20 minutes to 2 hours. In this way, large amounts of hemicellulose in herbaceous plant fibers can be hydrolyzed, and the gained dissolvable sugar can reach the maximum output.

(5) Pretreatment with water—adding pressure/temperature relief: in order to realize the inflation, separation and breakage of the cross linkages in the raw materials of xylem fibers during the pretreatment of herbaceous plant fibers with alcohol, liquid ammonia and alkali added together and under the high temperature and high pressure, in order to make the xylogen structures completely destroyed, and allow for the gained dissolvable sugar to be released and degraded, a transient decompression and transient cooling system is used along with real-time, water-adding temperature relief to attain the effect of real-time contraction and achieve final separation in the process of pretreatment.

(6) Recovery of organic solvents: alcohol, liquid ammonia, and dilute alkali NaOH are used in the process of heating and washing. This process includes washing the solid herbaceous plant fibers pretreated under high temperature and high pressure to avoid the re-separation of xylogen and xylan dissolved under the conditions of pretreatment. In this way, the cellulose, hemicellulose, and xylogen in herbaceous plant fibers can come into initial degradation. In order to reduce cost and protect the environment during production, all solvents used in the pretreatment must be recovered. The precise ultrafiltration system will eliminate solvents from the production system; it is necessary to recover them by such means, because organic solvents may restrain the growth and multiplication of microorganisms and affect enzymic hydrolysis and fermentation.

(7) Microorganic enzymic hydrolysis: the enzymic hydrolysis of cellulose is completed with the effective compound cellulase. The hydrolyzing materials include reducing sugar, biose, and xylose containing glucose. Compared with the process of using acid or alkali, the cost of enzymic hydrolysis is much lower, as it can be completed under relatively mild conditions (pH 4.0 to 4.6 and temperature 40 to 55° C.). In addition, the problem of corrosion will not arise. The prepared cellulase is generally a mixture of several enzymes. The hydrolysis process involves in at least three kinds of main cellulase:
endo β-glucanase (EG endo-1, 4-D-glycoside hydrolase, or EC3.3.1.4), mainly used in the region with low crystallinity in cellulose to generate short-chain molecules randomly;
exo-β-glucanase or cellase (CBH1, 4-β-D-glucan, cellose hydrolase, or EC3.2.1.91), used for further molecule degradation in the random short carbon chains to eliminate cellose; and
β-glucosidase (EC3.2.1.21) can degrade cellose into glucose.

Many coenzymes can also have effects on hemicellulose, such as glucosiduronate enzyme, acetyl enzyme, xylanase, β-xylanase, galactomannan enzyme, and glucomannan enzyme.

During enzymic hydrolysis, cellulose is degraded by cellulase into reducing sugar, which can be fermented by yeasts or bacteria into alcohol.

When alcohol is produced using cellulase to hydrolyze the cellulose in herbaceous plant fibers, the effect of cellulase is relatively slow because their substrate is a complex, insoluble, and semi-crystal structure. In addition, cellulase also requires comparatively high activity and the synergistic effect of relevant endo-β-glucanase, exo-β-glucanase, and β-glucosidate in order to completely convert cellulose into glucose.

When used to produce bio-alcohol to develop enzymes, cellulase is not only required to improve the stability of enzymes but also to increase the effective activity of enzymes under the technical conditions of pretreatment.

High-content xylogen can hold back the penetration of enzymes. Moreover, the activity of cellulase will be restrained by cellose and glucose, because they are strong cellulase inhibitors. Hence, several methods will be developed to reduce their inhibitory action, including use of high-concentration enzyme preparation as well as concurrent saccharification and fermentation.

After recovering the organic solvents, control the water temperature at 40 to 60° C. and adjust the pH value to between 3.0 and 6.0. Add 1% of the high-efficiency, high-concentration compound cellulase into the degradation solution of pretreated herbaceous plant fibers for enzymolysis. The enzymolysis process lasts about 10 hours, during which the raw materials of herbaceous plant fibers are degraded by compound cellulase into the reducing sugar, which can be fermented into alcohol by yeasts. Reducing sugar, biose, and xylose containing glucose are also produced in the enzymolysis process.

After the enzymolysis process completes, the fermentation process begins when the sugar concentration reaches 10 to 12 degrees as detected by the saccharometer. The diastase enables the starch molecules of glucose in the reducing sugar to decompose the β-1,4-glucoside bond from the non-reducing end to produce glucose and enable it to slowly hydrolyze β-1,6-glucoside bond to convert it into glucose. Control the water temperature at between 35 and 50° C. and adjust the pH value to between 3.0 and 6.0. Then, add the prepared compound yeasts to start the fermentation process, during which the prepared ammonium sulfate and phosphoric acid are added at the same time. The temperature in the fermentation tank is maintained at between 30 and 40° C. The fermentation process lasts for 50 to 80 hours. When the alcohol concentration reaches between 18 and 25 degrees as detected by the spectrophotometer, it will come into the preliminary process of refining alcohol from distillate spirits. When the alcohol concentration reaches between 35 and 50° C. after repeating the cooking and refinement, it will be sent to the alcohol tower for further refinement until the alcohol concentration reaches 95° C. During the whole process, simple manual management and automatic management can be applied.

### EXAMPLE

Grind the raw materials into a size of 100-180 Mesh, 110-170 Mesh, 120-160 Mesh or 130-150 Mesh.

The steam temperature is 100-250° C., 110-240° C., 120-230° C., 130-220° C., 140-210° C., 150-200° C., 160-190° C., or 170-180° C.; the pressure is 2-6 Mpa or 3-5 Mpa.

Enzymolysis process: the liquid temperature is between 40 and 60° C. or between 45 and 55° C.; pH value is between 3.0 and 6.0 or between 4.0 to 5.0; the fermentation temperature is between 30 and 40° C. or approximately 35° C.; the fermentation time is 50 to 80, 55 to 75, or 60 to 70 hours; the added yeast concentration is 3 to 8%, 4 to 7%, or 5 to 6%.

The equipment involved in this invention uses widely recognized technologies. Certain values (e.g. proportion of ingredients) not described in this invention should be known by the technicians in this field.

What is claimed is:

1. A method of converting herbaceous plant fibers into fuel alcohol, comprising the following steps:
(a.) grinding raw materials of herbaceous plants to the size between 100 and 180 Mesh, and loading them in a reaction tank;
(b.) adding CO₂ gas and applying supersonic waves;
(c.) adding a liquid mixture of alcohol: liquid ammonia: water in a ratio of 30-40%:6-10%:50-64% by weight to the raw material in a proportion of between 1:1.5 and 1:3 of the raw materials to the liquid mixture by weight; adding dilute alkali NaOH in a proportion of 3-12% of alkali to the raw materials by weight, and stir the mixture;
(d.) boiling the mixture for 20 minutes to 2 hours under the temperature of 100-250° C. and the pressure of 2-6 MPa;
(e.) reducing the pressure to normal atmospheric pressure and reducing the temperature to 30-40° C.;
(f.) optionally, recovering the organic liquids;
(g.) under the temperature of 40-60° C. and pH between 3.0 and 6.0, adding aqueous suspension of cellulase comprising endo-β-glucanase, exo-β-glucanase and β-glucanase which account for 0.5-3% of the liquid weight, and allowing a reaction to take place for 8-12 hours; and
(h.) under the temperature of 40-60° C. and pH between 3.0 and 6.0, adding aqueous suspension of Candida mycoderma, Rhizopus oryzae and dry yeast which account for 3-8% of the liquid weight; optionally further adding ammonium sulfate and phosphoric acid; keeping the temperature at 30-40° C. and allowing reaction to take place for between 50 and 80 hours.

2. The method of claim 1, wherein when the alcohol concentration reaches between 18 and 25 degrees, the reaction mixture will be subjected to a preliminary process of refining alcohol from distillate spirits.

3. The method of claim 1, wherein when the alcohol concentration reaches between 35 and 50 degrees, the reaction mixture is sent to an alcohol tower for further refinement until the alcohol concentration reaches 95 degrees.

4. The method of claim 1, wherein the cellulase in step (g.) further comprises β-Glucoside acid enzyme, transacetylase, xylanase, β-xylanase, galactomannan enzyme and glucomannan enzyme.

5. The method of claim 1, further comprising transient pressure relief and real-time water-adding temperature relief.

6. The method of claim 1, wherein step (f.) further comprises a pretreatment using an alcohol which is different from the fuel alcohol, liquid ammonia and dilute alkali NaOH.
